(19) United States
(12) Patent Application Publication
(10) Pub. No.: US 2023/0225911 A1
(43) Pub. Date: Jul. 20, 2023

(54) ABSORBENT ARTICLE HAVING REAGENTS FOR DETECTING MEDICAL CONDITIONS

(71) Applicant: BabyRx LLC, New York, NY (US)

(72) Inventor: Anastasia SHORR, New York, NY (US)

(21) Appl. No.: 18/099,487

(22) Filed: Jan. 20, 2023

Related U.S. Application Data
(60) Provisional application No. 63/301,253, filed on Jan. 20, 2022.

(51) Int. Cl. A61F 13/49; A61F 13/511; A61F 13/537; A61F 13/15; A61F 13/42; A61B 10/00

(52) U.S. Cl. CPC A61F 13/49; A61F 13/511; A61F 13/5376; A61F 13/15252; A61F 13/42; A61B 10/00; A61B 2010/0006

(57) ABSTRACT

An absorbent article is worn to receive discharged urine of the wearer. The article includes a liquid-permeable top sheet, an intermediate liquid-absorbent layer, a bottom sheet including at least one indicator and a liquid-impermeable backing layer. Each of the at least one externally visible indicator is coated or impregnated with at least one chemical reagent facing the liquid-absorbent layer. Each of the at least one chemical reagent is configured to undergo a change in color or shade from a plurality of different possible color or shade changes of the reagent upon contact with the discharged urine. Each color or shade is associated with a corresponding level of an analyzed component or with the presence or absence of the analyzed component and the change in color or shade of the at least one chemical reagent is externally visible on the indicator through the bottom sheet and the backing layer.

### CROSS-REFERENCE TO RELATED APPLICATIONS

This application claims priority from similarly-titled U.S. Provisional Patent Application No. 63/301,253, filed Jan. 20, 2022, the entire contents of which are incorporated by reference herein.

### BACKGROUND OF THE DISCLOSURE

The present disclosure is generally directed to a diaper and other such absorbent articles, which include one or more chemical reagents for detection of a variety of medical conditions.

Detection of various substances and amounts of various substances in urine can provide information about the overall health of an individual, including indicating if the individual is suffering from any health problems or medical conditions. While urine testing may be readily performed on human adults (e.g., by voluntary collection of a urine sample), such testing cannot be readily performed on some individuals, such as infants and children, elderly adults, non-ambulatory individuals and the like.

Therefore, it would be beneficial to provide an apparatus, such as a diaper or other absorbent article, having one or more chemical reagents that indicate the existence of a medical condition based on detection of substances or atypical levels of substances in an individual's urine.

### BRIEF SUMMARY OF THE DISCLOSURE

Briefly stated, one aspect of the present disclosure is directed to a wearable absorbent article configured to receive discharged urine of the wearer. The article includes a liquid-permeable top sheet, an intermediate liquid-absorbent layer, a bottom sheet including at least one indicator, and a liquid-impermeable backing layer. Each of the at least one externally visible indicator is coated or impregnated with at least one chemical reagent facing the liquid-absorbent layer. Each of the at least one chemical reagent is configured to undergo a change in color or shade from a plurality of different possible color or shade changes of the reagent upon contact with the discharged urine. Each color or shade is associated with a corresponding level of an analyzed component or with the presence or absence of the analyzed component and the change in color or shade of the at least one chemical reagent is externally visible on the indicator through the bottom sheet and the backing layer.

### BRIEF DESCRIPTION OF THE SEVERAL VIEWS OF THE DRAWINGS

The following description of an embodiment of the disclosure will be better understood when read in conjunction with the appended drawings. It should be understood, however, that the disclosure is not limited to the precise arrangements and instrumentalities shown. In the drawings:

FIG. 1 is an exploded, top perspective view of an absorbent article of the present disclosure; and

FIG. 2 is a schematic, bottom plan view of the absorbent article of FIG. 1.

### DETAILED DESCRIPTION OF THE DISCLOSURE

Certain terminology is used in the following description for convenience only and is not limiting. The words “lower,” “bottom,” “upper” and “top” designate directions in the drawings to which reference is made. The words “inwardly,” “outwardly,” “upwardly” and “downwardly” refer to directions toward and away from, respectively, the geometric center of the article, and designated parts thereof, in accordance with the present disclosure. Unless specifically set forth herein, the terms “a,” “an” and “the” are not limited to one element, but instead should be read as meaning “at least one.” The terminology includes the words noted above, derivatives thereof and words of similar import.

It should also be understood that the terms “about,” “approximately,” “generally,” “substantially” and like terms, used herein when referring to a dimension or characteristic of a component of the disclosure, indicate that the described dimension/characteristic is not a strict boundary or parameter and does not exclude minor variations therefrom that are functionally similar. At a minimum, such references that include a numerical parameter would include variations that, using mathematical and industrial principles accepted in the art (e.g., rounding, measurement or other systematic errors, manufacturing tolerances, etc.), would not vary the least significant digit.

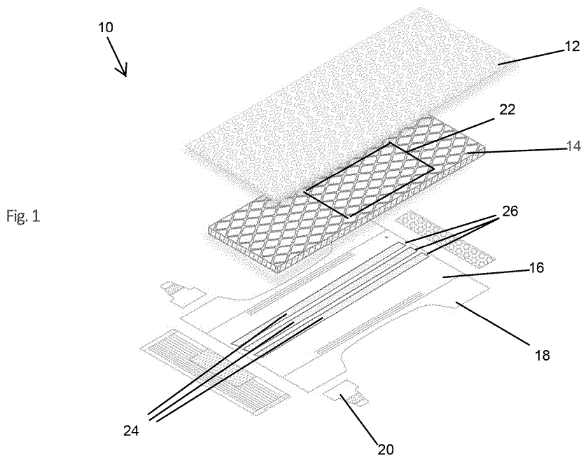

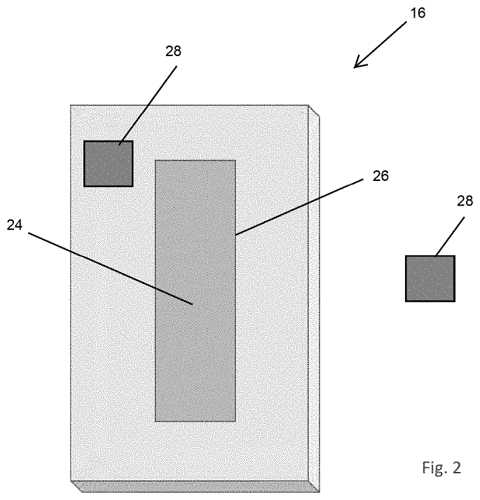

Referring to the drawings in detail, wherein like numerals indicate like elements throughout, there is shown in FIGS. 1-2 a wearable absorbent article, generally designated 10, such as a diaper, in accordance with an embodiment of the present disclosure. Referring to FIG. 1, there is shown an exploded view of the different layers of the diaper 10, as described in greater detail below. As should be understood by those of ordinary skill in the art, the diaper 10 may be of any type which is currently publicly available or which is yet to be developed, with the modification that a bottom sheet 16 of the diaper is provided with one or more chemical reagents, as described in greater detail below. Generally, the diaper 10 includes a liquid-permeable top sheet 12, an intermediate liquid-absorbent pad, core or layer 14, the bottom sheet 16, and a liquid-impermeable backing film or layer 18. The diaper generally also includes a mechanism 20 for placing the diaper in a closed, use position secured to the wearer (not shown). Non-limiting examples of such a mechanism 20 include an adhesive tape, Velcro®, a combination thereof and the like.

The liquid-permeable top sheet 12, liquid-absorbent layer 14, bottom sheet 16 and liquid-impermeable backing layer 18 may be made from any materials known or commonly used for forming these respective layers in diapers. For example, the liquid-absorbent pad, core or layer 14 may be made of a super-absorbing polymer such as sodium polyacrylate, fluff made from wood pulp fibers, corn/wheat-based materials, paper, cloth, or any other fibers currently known or that later become know which have liquid absorbing properties, or a mixture of any of these materials. The liquid-permeable top sheet 12 may be made from woven, non-woven, or porous formed-film polyethylene or polypropylene materials. The bottom sheet 16 may be made from a non-woven layer. The liquid-impermeable backing film or layer 18 may be made from polyethylene, a composite film, a hybrid or the like. In one configuration, the diaper 10 is biodegradable and the liquid-permeable top sheet 12 and/or bottom sheet 16 is/are made from a non-woven layer of natural fibers, e.g., plant-based fibers, such as cotton, hemp, flax, ramie, bamboo and the like. In one configuration, the bottom sheet 16 and liquid-impermeable backing film or layer 18 are an integral component.

Optionally, the diaper 10 may further include an acquisition layer 22 sandwiched in between the liquid-permeable top sheet 12 and the liquid-absorbent layer 14. The acquisition layer 22 may be made of a material or combination of materials that accelerate the diffusion of liquid from the top sheet 12 into the absorbent layer 14, and thus improve the wicking effect of the diaper 10 and removal of moisture from contact with the wearer's skin (not shown), ensuring that the wearer stays drier.

The bottom sheet 16, and more particularly the non-woven bottom sheet 16, of the diaper 10 includes at least one chemical reagent 24. Each chemical reagent 24 is provided in the bottom sheet 16, and more particularly in a region of the bottom sheet 16 facing, e.g., underlying, or proximate to the liquid-absorbent core 14 and most likely to be contacted by the wearer's discharged urine. Each chemical reagent 24 serves to detect a substance in the urine whose presence or concentration is atypical, and thus detect a medical condition of the wearer. In one configuration, each chemical reagent 24 is configured to undergo a change in color upon detection of a substance in the urine whose presence or concentration is atypical, thus providing a clear and visible indication to caretakers and users of one or more possible abnormalities or medical conditions.

In configuration, as shown in FIG. 1, a plurality of chemical reagents 24 are provided, e.g., in a structured or predetermined arrangement, along the bottom sheet 16 so as to enable the detection of various different medical conditions from which the wearer of the diaper 10 may be suffering. In one configuration, an indicator 26 which is externally visible to users and caretakers is provided for each respective chemical reagent in the bottom sheet 16. The indicator 26 may be a material form, such as a strip, sheet or particles, which is/are coated or impregnated with a chemical reagent 24 or mixture of chemical reagents 24 and embedded and immobilized in the diaper 10. In an additional or alternative configuration, the chemical reagents 24 may be provided in predetermined areas of the bottom sheet 16 in the form of powder, or in the form of fibers coated or impregnated with the reagents 24 and woven throughout predetermined areas of the bottom sheet 16.

As shown in FIG. 2, which is a bottom plan view of the diaper 10 in an open and flat position, each indicator 26 is preferably externally visible to users and caretakers through the bottom sheet 16 and backing film 18. Thus, any color changes to each indicator 26 can be assessed without taking the diaper 10 off the wearer. In one configuration, where a plurality of medical conditions are to be detected by the apparatus, a plurality of distinct indicators 26 may be present, each indicator 26 being configured to detect a different medical condition. In one configuration, a single strip containing a series of discrete chemical reagent 24 coated or impregnated indicators 26 that are separated by buffers or dividers may be provided, such that each discrete section is configured to change color and indicate detection of a different medical condition or is configured to change color to indicate detection of a specific level of a substance. In one configuration, a strip or sheet comprising a plurality of distinct chemical pads or reagents which react (change color) when contacted with discharged urine may be embedded or otherwise secured within the diaper 10, such as in the bottom sheet 16. For ease of use, each indicator or chemical pad 26 may be labelled, such as with the name, abbreviation or other identifier, of the substance, characteristic or medical condition to be detected thereby.

Examples of the medical conditions that may be detected by the present disclosure include, but are not limited to, urinary tract infections, kidney stones or other kidney disorders or infections, diarrhea, metabolic syndromes, glucosuria, dehydration and the like.

The chemical reagents 24 are preferably configured to change color in 6 minutes or less, such as 5 minutes or less, 4 minutes or less, 3 minutes or less, 2 minutes or less, or 1 minute or less, such as within between approximately 1 second and approximately 1 minute, upon contact with discharged urine. As such, detection of any potential harmful medical conditions can be achieved quickly and easily. It will be understood by those skilled in the art that the discussion herein of color changes which occur “upon contact with the urine” or “upon contact with discharged urine” encompass the aforementioned durations of time (e.g., anywhere between approximately 1 second and approximately 6 minutes).

Examples of the chemical reagents 24 that may be used include, but are not limited to, reagents 24 known to indicate the presence of substances or certain levels of substances in discharged urine, such as white blood cells, nitrites, calcium, glucose, albumin or microalbumin, creatine, ascorbate, blood and the like, by a change in color upon contact with discharged urine.

In one exemplary embodiment, at least one reagent 24 may be employed which changes to different colors or shades to indicate the presence of white blood cells in the discharged urine, which may be indicative of a urinary tract infection. Examples of such reagents 24 include, but are not limited to, derivatized pyrrole amino acid ester, diazonium salt, and mixtures thereof.

In one exemplary embodiment, at least one reagent 24 may be employed which changes to different colors or shades to indicate the presence of nitrites (a bacterial metabolism by-product of nitrates) in the discharged urine, which may be indicative of a urinary tract infection. Examples of such reagents 24 include, but are not limited to, p-arsanilicacid; 0.9% N-(1-Naphthol) ethylenediamine, tetrahydro benzoquinoline, and mixtures thereof.

In one exemplary embodiment, at least one reagent 24 may be employed which changes to different colors or shades to indicate the presence of ketones or an abnormal level of ketones in the discharged urine, wherein high ketone levels may be indicative of diabetic ketoacidosis (DKA). An example of such a reagent 24 is sodium nitroprusside.

In one exemplary embodiment, at least one reagent 24 may be employed which changes to different colors or shades to indicate the presence of bilirubin in the discharged urine, which may be indicative of a gallbladder and liver issues. An example of such a reagent 24 is dichlorobenzene diazonium salt.

In one exemplary embodiment, at least one reagent 24 may be employed which changes to different colors or shades to indicate a normal calcium level (e.g., approximately 100 to approximately 300 mg/day), a high calcium level (e.g., above 300 mg/day) which may be indicative of chronic kidney disease, kidney stones, sarcoidosis and the like, or a low calcium level (e.g., less than 100 mg/day).

In one exemplary embodiment, at least one reagent 24 may be employed which changes to different colors or shades to indicate the presence of glucose in the discharged urine. A color change in such a reagent 24 indicates the presence of glucose in the discharged urine and is therefore indicative of a condition known as glycosuria. An example of such a reagent 24 is glucose oxidase/peroxidase.

In one exemplary embodiment, at least one reagent 24 may be employed which changes to different colors or shades to indicate protein levels in the discharged urine. A color change or change in shade of color in such a reagent 24 upon contact with urine indicates the presence of protein, particularly albumin or microalbumin, or atypical protein levels in the discharged urine and is therefore indicative of a condition known as proteinuria or of kidney damage. Examples of such a reagent include, but are not limited to, sulfonephtalein dye, tetrabromophenol blue and 3′, 3″, 5′, 5″-tetrachlorophenol, 3,4,5,6-tetrabromosulfonphthalein, and mixtures thereof.

In one exemplary embodiment, at least one reagent 24 may be employed which changes to different colors or shades to indicate the presence or abnormal levels of creatinine in the discharged urine, which is indicative of poor kidney function. Examples of such a reagent 24 include, but are not limited to, copper sulfate and benzidine, and mixtures thereof.

In one exemplary embodiment, at least one reagent 24 may be employed which changes to different colors or shades to indicate the presence of blood in the discharged urine. A color change in such a reagent 24 indicates the presence of blood in the discharged urine and is therefore indicative of a condition known as hematuria. An example of such a reagent is tetramethylbenzidine/peroxidase.

In one embodiment, at least one reagent 24 may be employed which changes to different colors or shades to indicate the presence or abnormal levels of ascorbate (ascorbic acid) in the discharged urine. High levels of ascorbate can interfere with and lead to false results in other urine tests. Examples of such a reagent 24 include, but are not limited to, 2,6-dichlorophenol indophenol sodium salt.

Other examples of chemical reagents 24 that may be employed include, but are not limited to, reagents 24 known to indicate different levels of pH, specific gravity and the like, by a change in color upon contact with discharged urine. More particularly, examples of the chemical reagents 24 that may be used include, but are not limited to, reagents 24 known to indicate atypical levels of pH, specific gravity and the like, by a change in color upon contact with discharged urine.

In one exemplary embodiment, at least one reagent 24 may be employed which changes to different colors or shades to indicate a normal pH level (e.g., close to 7), a high (alkaline) pH level which may be indicative of an infection caused by Escheriachia Coli or Klebsiella Pneumoniae, or a low (acidic) pH level which may be indicative of an infection caused by Proteus Mirabilis or Pseudomonas Aeruginosa. Examples of such a reagent 24 include, but are not limited to, methyl red and bromothymol blue. Whereas conventional indicators using such reagents function in a binary mode, simply indicating wetness or dryness, the chemical reagents 24 of the present disclosure advantageously provide a scale wherein each color or shade is associated with a specific level of a component or with the presence or absence of a component.

In one exemplary embodiment, at least one reagent 24 may be employed which changes to different colors or shades to indicate a specific gravity level in the discharge urine. High specific gravity indicates dehydration. Examples of such reagents 24 include, but are not limited to, methyl vinyl ether/maleic anhydride and bromothymol blue, and mixtures thereof.

As should be understood by those of ordinary skill in the art, urinalysis indicators include a dye that changes in appearance, e.g., changing color, appearing, disappearing, or the like on contact with discharged body fluids. The dye that affects this change is one component of the urinalysis indicator, and can change appearance based on pH, temperature, moisture, and proteins detected in the discharged body fluids.

To detect leukocytes, or white blood cells, two or more reagents 24 may be employed in the indicator 26 formulation, with two key reagents 24 being ester and a diazonium indicator. Reagents 24 based on proteolytic substrates are used to detect the presence of proteases in discharged body fluids. The binding site that the protease recognizes is replicated in the synthetic proteolytic, which when cleaved by the protease, releases a signal generating product that affects a noticeable change in the indicator 26. The presence of leukocytes is inferred from whether the exposed indicator 26 reflects light at characteristic wavelengths of the products that are produced from these proteolytic reactions. Typically, the incubation time for these reactions ranges from between approximately thirty seconds and approximately two minutes after the indicator 26 is exposed to body fluids.

There are several options for leukocyte reagents 24 which may provide a method of measuring the leukocyte content of urine samples. One composition employs an alanine compound (PPTA; 2-hydroxy -5 -phenyl -pyrrole -N-tosyl -L- alanine ester) as a synthetic substrate for leukocyte esterase, which cleaves PPTA, after which the cleaved product reacts with a diazonium indicator (DNSA; 1-diazo-2-naphthol-4-sulfonic acid) to produce a color change. The color change is visible in the 520-570 nm wavelength range.

With respect to esterase substrates and diazonium indicators 26, esterase substrates can include esters of the following categories: indoxyl acetate, indoxyl butyrate, indoxyl laureate, indoxyl stearate, and indoxyl ester. Lactate esters can also be used as well as thiazole esters, pyrrole esters, thiophene esters, napthyl esters, pheoxyl esters, and quinolinyl esters. Examples include 3-(N-tosyl-L-alaninyloxy)-5-phenylthiophene, 3 - (N-to syl-L- alaninyloxy)-1-methyl-5-phenylpyrrole, 3- (N-to syl-L- alaninyloxy)-5- (p-chlorophenyl) pyrrole, 3 - (N-to s yl-L-alaninyloxy)-5-phenylpyrrole, 1-naphthyl N-acetyl-DL-alanine, 1-naphtyl N-acetyl-L-alanyl-L-alanyl-L-alanine and 1-naphthyl butyrate.

Other potential diazonium indicators 26 include 1-diazo-2-naphthol-4-sulfonate, 1-diazophenyl-3 -c arbonate, 4-diazo-3 -hydroxy-l-naphthyls ulfonate (DNS A), 4-diazo-3 -hydroxy-7-nitro- 1-naphthylsulfonate (NDNS A), 4-diazo-3-hydroxy-1,7-naphthyldisulfonate, 2-methoxy-4- (N-morpholinyl) benzene diazonium chloride, 4-diazo-3-hydroxy-7-bromo- 1-naphthylsulfonate and 4-diazo-3-hydroxy-7- [1, oxopropyl]-1-naphthylsulfonate.

Other components of the dyes for the reagents 24 include solvents, ion strength agents, buffers, surfactants, polymers, and enzyme activators. Both polar and nonpolar solvents are used to help all components mix evenly. Water and acetone are a common pair of solvents used together. Boric acid is a useful buffer that ensures that the pH stays within functional ranges for the reagents. Sodium chloride, calcium carbonate, and sodium bisulfate are examples of salts that act as ion strength agents that reduce the ion concentration of the surrounding solution. Decanol is a suitable enzyme activator that helps ensure that the leukocyte esterase can access ester groups properly and cleave them.

To detect nitrites, which are signs of potential urinary tract infections, three or more ingredients may be employed to create a reliable detector, with three key reagents being a diazotizing amine, a coupling agent, and a buffer. One non-limiting example is: 4,4′ sulfonyldianiline (as a diazotizing amine), N-(1-naphthyl)-ethylenediamine (coupling agent), and organic polycarboxylic acid (as buffer). Examples of diazotizing amines that can be used also include: aniline; 4-chloroaniline; 4-bromoaniline; 2,4,6-tribromoaniline; 2,4,6-trichloroaniline; alpha -trifluoro-m-toluidene; Ortho-toluidine; m- and p-aminophenols; Ortho-tolidine; Sulfanilamide, p-aminobenzoic acid; 1 -amino- 8-hydroxynaphthalene-3 ,6-dis ulfonic acid; Amino acetanilide ;

Aminophenyl ether; p-arsaline acid; 4-amino- 1-naphthalenecarbonitrile. Examples of suitable coupling agents for creating these solutions include: 8-hydroxygallolidine, N, N-dimethylaniline; Methylenedianiline; Benzidine; B enzoquinoline; Aminoquinoline; m-phenylenediamine; alpha -trifluoro-m-toluidene; Ortho-toluidine; m-aminophenol; Ortho-tolidine; Derivatives thereof.

Multiple options may be employed for buffers that can regulate the nitrite reaction. Non-limiting examples of suitable buffers in this application include inorganic acids, sulfonic acids such as 2- [N-morpholino] ethanesulfonic acid (“MES”), carboxylic acids, and polymeric acids. More Specific examples of suitable inorganic acids are hydrochloric acid, nitric acid, phosphoric acid and sulfuric acid. Specific examples of suitable carboxylic acids are citric acid, glycolic acid, lactic acid, acetic acid, maleic acid, gallic acid, malic acid, succinic acid, glutaric acid, benzoic acid, malonic acid, salicylic acid, gluconic acid, and mixtures thereof.

Binders or binding agents fix a dye, colorant, or combination of colorants within the material to stop leaching of the dyes into adjacent regions or layers of the absorbent substrate. For enhanced visibility and clarity of the color change to an observer, it is advantageous to “lock” the dye within the binder or adhesive before and after contact with discharged body fluids like urine and feces. The binding agency is also key to ensuring that the dye and other components of the urinalysis indicator adhere to the substrate itself, which may be a component of the absorbent article such as the absorbent layer 14 or the bottom sheet 16. Non-limiting examples of binders that are suitable for this application include hot melt adhesives or solvent-based binding matrices for the urinalysis indicator 26. The binding matrix includes components such as hot melt adhesives, wetting agents, base polymers, tackifiers, waxes, solvents, plasticizers, stabilizers, anti-oxidants, and other additional ingredients.

Potential binders include, but are not limited to, rosins, gum rosin, distilled rosin, hydrogenated rosin, polymerized rosins, pentaerythritol rosin esters, wood rosin, tall-oil rosin, dimerized rosin, polymerized rosin, rosin esters, modified styrene-acrylic polymers and their salts, styrenated terpenes, polyterpene resins, terpene phenolics, and combinations thereof.

It may be advantageous for the initial state of the urinalysis indicator 26, prior to contact with discharged body fluids or other liquids, to be white and lacking in coloration. This can be accomplished through the use of synthetic ingredients designed to be white in their dry state. Natural rosins tend to be yellow in color in the dry state, and are usually naturally derived from trees. However, in some cases, adding white opacifiers like sodium aluminum silicate, titanium dioxide, and various sulfates and other minerals may be beneficial. These opacifiers can reduce or hide the yellow color of natural rosins, making changes in color of the dye easier for observers to detect. Opacifiers generally make up between approximately 0% (if not used) and approximately 1% of the urinalysis indicator by weight (if used). It is also possible to omit the use of opacifiers, and use natural rosins that have reduced natural yellow coloration when in their dry state.

The binders “lock” the dyes and other components when they are in their dry, and initial color state. Operation depends on the binder, the colorant, other additives, and how they interact on a molecular level. Generally, the binder immobilizes the other components of the urinalysis indicator through one or more of the following intermolecular and interatomic forces: polar covalent bonding, nonpolar covalent bonding ionic bonding, hydrogen bonding, weak intermolecular forces including Van der Waals forces, dipole-dipole forces, dispersion forces, and any and all combinations of these forces. Effective ranges of the percent composition of binders in urinalysis indicators varies, but can include percent compositions ranging from between approximately 10% and approximately 90%, such as between approximately 20% and approximately 65% binders in their urinalysis indicators, by weight.

It may be useful to use multiple binding agents in certain formulations. In such cases, the second binder may complement the first binder in preventing color leaching, and ensuring the urinalysis indictor adheres to the desired substrate. In some cases, the binder may perform only one of these functions, leaving it to the primary binder to be primarily responsible for the other function. Non-limiting examples of secondary binders that may be useful complements to primary binders include binders such as acrylic-based solvents like acrylates/ethylhexyl acrylate copolymers; sodium acrylate/sodium acryloyldimethyl taurate copolymer; acrylates/Octyl acrylate copolymer; ammonium polyacrylate. Organic solvents may include ethanol, propanol, butanol, acetone, tetrahydrofuran, benzene, toluene and acetonitrile. Water-based resins also make good binding agents, and examples include polyamide, cellulose derivatives, an acrylic polymer or a polyol, e.g. a water soluble resin selected from the group consisting of polyvinylpyrrolidone, polyvinyl alcohol, carboxymethylcellulose, poly(2-ethyl-2-oxazoline), polymers (homopolymers and copolymers) based on acrylic acid, polymers (homopolymers and copolymers) based on methacrylic acid), and polymers (homopolymers and copolymers) based on acrylamide and any combination thereof.

Hot melt adhesives may be used as binders in urinalysis indicators, and include a wide range of base polymers that are effective binders and adhesives. Hot melt adhesives contain many components, which may include, but are not limited to, wetting agents, tackifiers, waxes, solvents, plasticizers, stabilizers, anti-oxidants, and additional ingredients. Base polymers may include polyvinylpyrollidone/vinyl acetate copolymers, polyethylene, ethylene-methyl acrylate, ethylene n-butyl acrylate, polypyrrolindone homopolymers, ethylene-vinyl acetate (EVA) copolymers, ethylene/acrylic acid copolymers, styrenic block copolymers, vinyl pyrrolidone homopolymers, polyamides, kraton polymers, ethylene/acrylic acid co-polymers, amorphous propylene/ethylene, amorphous propylene, ethylene/butane, styrene block copolymers, styrene/acrylic polymers, polycarbonates, synthetic polyisoprene, polybutadiene rubber, chloroprene rubber, polyacrylic rubber, polyether block amids, polymers of acrylates, EVA, epoxy resins, fluoropolymers, hydrocarbon resins, phenols, polyesters, olefins, polyurethanes, silicones and functionalized silicones, polystyrene, polyvinyls, oxidized polyethylene, polybutene-1, amorphouse polyolefines, and epichlorohydrin rubber.

Wetting agents such as mineral oils, alkanes, polyethylene glycols, glycols, glycerine, isoparaffins, silicone fluids, and water may be used to reduce viscosity or increase the solubility of other additives in the urinalysis indicator solution. Both minearal and polymerized waxes may be used in hot-melt adhesives. Non-limiting examples include microcrystalline waxes, polyethylene waxes, highly branched polymer waxes, beeswax, soywax, polyethylene glycol waxes, polymethylene waxes, ozokerite, ceresin, water soluble waxes, silicone waxes, polyethylene waxes, and paraffin waxes. Solvents ensure different additives do not precipitate out of solution, and hence out of the urinalysis indicators. Non-limiting examples of solvents include paste, alcohols, mineral oils, silicone fluids, alkanes such as hexane, xylenes, polyethylene glycols such as PEG-200, glycosls, adhesive material, thixotripic material, and esters. Alcohol solvents may include ethanol, methanol, butanol, iso-propyl alcohol, n-propyl alcohol, and ester alcohols. Acetates are also beneficial, non-aqueous solvents, and include n-propyl acetate, isopropyl acetate, and others. Aqueous solvents are solvents that include water. Non-limiting examples of aqueous solvents include water, detergent solutions, alkaline and acidic water-based solutions.

Non-limiting examples of plasticizers, which make adhesives more flexible and pliable, include triethoxycaprylysilane, castor oil, alkyl benzoate, polyglyceryl-4 isostearate, antistatic agents, rosins and derivatives, alkyl citrates, phthalates, phthalate esters, paraffin oils, glyceryl behenate, propylene carbonate, hexyl laurate, esters such as isopropyl myristate, and other viscosity modifiers. Beneficial surfactants for urinalysis indicators include, without limitation, alkoxylated glyceryl and polyglyceryl alkylates such as PEG-30, alkoxylated hydrogenated castor oil, lanolin, sugar esters, poloxamers, polysorbates, ethoxylated sorbitan esters, tergitol, ethoxylated alcohols, fatty alcohols, block copolymers such as propylene glycol-ethylene glycol block copolymers, polyoxypropylene-polyoxyethylene block copolymers, and poly(ethylene oxide)-block-poly(propylene oxide) copolymers. Other surfactants include linear alkyl alcohol ethoxylate, propylene oxide block copolymers, polyalkylene oxide block copolymer, fatty acid esters, ethoxylates, propoxylates, diethylene glycol, alkyl ethoxylate sulfonates, glycerol monostearate, and other anionic and cationic surfactants.

Antioxidants include, for example, without limitation, phenol base antioxidants such as 2,6-di-tert-butyl-p-crresol, 2,2′-methylenebis(4-methyl-6-tert-butylphenol), 4,4′-butylidenebis(3-methyl-6-tert-butylphenol), 4,4′-thiobis(3-methyl-6-tert-butylphenol), 2,2′-thiobis(4-methyl-6-tert-butylphenol), stearyl-0-(3,5-di-tert-butyl-4-hydroxyphenol)propionate, tetrakis [methylene-3 -(3 ,5-di-tert-butyl-4-hydroxyphenyl)propionate] methane, triethylene glycol, bis [3 -(3 -tert-butyl-4-hydroxy methylphenyl)]propionate, 1,3,5-triethyl-2,4,6-tris(3,5-di-tert-butyl-4-hydroxybenzyl)benzene and 1,1,3-tris(2-methyl-5-tert-butylphenol)butane; amine base antioxidants such as phenyl-α-naphthylamine, phenyl-β-naphthylamine, N-phenyl-N′-cyclohexyl-p-phenylenediamine and N-isopropyl-N′-phenyl-p-phenylenediamine; phosphorus base antioxidants represented by triisodecyl phosphite and 2,2′-methylenebis(4,6-di-tert-butylphenyl)octyl phosphite; sulfur-containing antioxidants represented by 2,2′-thiobis(4-methyl-6-tert-butylphenol); and hydroquinone antioxidants represented by 2,5-di-tert-amylhydroquinone. In the present disclosure, the phenol base antioxidants are particularly preferably used.

An exemplary leukocyte dye may include the following components, in the following allowable ranges, and operative for the following functions, as shown in the table below.

| Component | Function | Allowable Range | |
|---|---|---|---|
| Water | Solvent | — | |
| NaCl | Ion Strength Agent | 1-30 | g/L |
| Bio-Terge AS40 | Surfactant | 0-4 | g/L |
| Boric acid | Buffer | 5-35 | g/L |
| PVP | Polymer | 5-50 | g/L |
| Acetone | Solvent | 200-400 | g/L |
| DMSO | Solvent | 10-60 | mL/L |
| 1-diazo-2-napthol-4-sulfonic | Diazonium Indicator | 0.050-0.5 | g/L |
| acid | | | |
| 2-hydroxy-5-phenyl-pyrrole- | Enzyme Substrate | 0.10-0.8 | g/L |
| N-tosyl-L-alanine ester | | | |
| Decanol | Enzyme Activator | 5-40 | mL/L |

An exemplary hot melt adhesive may include the following components, in the following allowable ranges, and operative for the following functions, as shown in the table below.

| | | Allowable Range |
|---|---|---|
| Component | Function | (wt %) |
| Ethylene Vinyl Acetate | Binder | 10-20% |
| Leukocyte Dye | Indicator | 0.10-0.50% |
| Phloxine B | Permanent | 0.05-0.1% |
| | Colorant | |
| Ethylene Acrylic Acid Co-polymer | Binder | 40%-50% |
| PEG-30 | Surfactant | 30%-45% |
| 2,6-di-tert-butyl-p-crresol | Antioxidant | 0.5%-2% |
| Isononyl Isononanoate | Wetting Agent | 1%-2% |

One, non-limiting, method of preparation is as follows: all components except the polymers and the dye components are added to a heated mixer. The mixer is heated to a temperature of 120° C. and mixed until homogenous. While maintaining mixing and heating at 120 ° C., the polymers are slowly added to the mixer, until dissolved. When the polymers have dissolved completely, add the dye components, and mix until homogenous again, which may take two hours or longer. Either maintain heating to safely prepare adhesive films with a draw-down wire or let the mixture solidify at room temperature for storage and future use. If a draw-down wire is used, the adhesive can be applied to substrates, for example, the bottom sheet 16 of the diaper 10, and serve as an indicator 26 of leukocytes in urine. Coating is performed using a bench scale hot melt coaster, which contains a slot die with one nozzle to produce one stripe on the bottom sheet 16. The coating weight should be between approximately 0.1 g/m/line and approximately 0.2 g/m/line.

Other methods of manufacturing include, but are not limited to, using propeller-blade mixers, sigma-blade mixers, and using lamination to allow the indicator 26 to prevent it from blocking while on the substrate. Ingredients may also be added at different points during the process, including but not limited to adding the surfactant last (after the dye is added).

The diaper 10 is designed such that the change in color of the chemical reagents in the bottom sheet 16 can be interpreted by individuals having little to no medical training, in order to detect one or more medical conditions. The diaper or other absorbent article 10 may be accompanied by a color chart 28 for each reagent present in the bottom sheet 16. The color chart 28 may be integrated with the diaper 10, separate from the diaper 10, or both. The color chart 28 preferably includes an indicator scale (e.g., a color gradient), wherein each color on the scale is associated with a specific level of a component or with the presence or absence of a component. Thus, an individual can compare the color of the reagent in the used diaper 10 with the color chart to determine if a harmful substance is present or if a harmful or atypical level of a substance is present in the wearer's urine. For at least some of the chemical reagents 24, no color chart 28 may be needed, as any change in color of a reagent 24 could mean the presence of a harmful medical condition.

It will, therefore, be appreciated by those skilled in the art that various modifications and alterations could be made to the disclosure above without departing from the broad inventive concepts thereof. Some of these have been discussed above and others will be apparent to those skilled in the art. For example, the present disclosure primarily disclosed a diaper, such as a disposable diaper, but it should be understood by those skilled in the art that the present disclosure is applicable to any absorbent article, such as a sanitary pad or liner, an incontinence pad or liner, and the like. It is understood, therefore, that this disclosure is not limited to the particular embodiments disclosed, but it is intended to cover modifications within the spirit and scope of the present invention, as set forth in the appended claims.

We claim:

1. A wearable absorbent article configured to receive discharged urine of a wearer, the article comprising:
a liquid-permeable top sheet;
an intermediate liquid-absorbent layer;
a bottom sheet including at least one indicator, each of the at least one externally visible indicator being coated or impregnated with at least one chemical reagent facing the liquid-absorbent layer, each of the at least one chemical reagent being configured to undergo a change in color or shade, from a plurality of different possible color or shade changes of the chemical reagent, upon contact with the discharged urine; and
a liquid-impermeable backing layer,
wherein each color or shade is associated with a corresponding level of an analyzed component or with the presence or absence of the analyzed component and the change in color or shade of the at least one chemical reagent is externally visible on the indicator through the bottom sheet and backing layer.

2. The wearable absorbent article of claim 1, wherein the liquid-permeable top sheet is a non-woven sheet.

3. The wearable absorbent article of claim 1, wherein the bottom sheet is a non-woven sheet.

4. The wearable absorbent article of claim 2, wherein the liquid-permeable top sheet and the bottom sheet are constructed of non-woven, natural fibers.

5. The wearable absorbent article of claim 1, wherein the article is biodegradable.

6. The wearable absorbent article of claim 1, wherein the bottom sheet and the liquid-impermeable backing layer are integrally formed.

7. The wearable absorbent article of claim 1, further comprising an acquisition layer in between the liquid-permeable top sheet and the liquid-absorbent layer, the acquisition layer being configured to accelerate diffusion of liquid from the top sheet into the absorbent layer.

8. The wearable absorbent article of claim 1, comprising a plurality of the indicators, each distinct indicator being coated or impregnated with a corresponding one of the chemical reagents.

9. The wearable absorbent article of claim 1, wherein the at least one indicator comprises one indicator coated or impregnated with a plurality of the spaced apart chemical reagents.

10. The wearable absorbent article of claim 1, wherein each of the at least one indicator is labelled with an identifier of the substance, characteristic or medical condition to be detected thereby.

11. The wearable absorbent article of claim 1, further comprising a color chart providing an indicator scale associating each color or shade with the corresponding level of the detected component or with the presence or absence of the detected component.

12. The wearable absorbent article of claim 11, wherein the claim chart is a standalone component.

13. The wearable absorbent article of claim 1, wherein the article is a diaper.
